(19) United States
(12) Patent Application Publication
(10) Pub. No.: US 2021/0150783 A1
(43) Pub. Date: May 20, 2021

(54) UNSUPERVISED LEARNING-BASED MAGNETIC RESONANCE RECONSTRUCTION

(71) Applicant: Siemens Healthcare GmbH, Erlangen (DE)

(72) Inventors: Simon Arberet, Princeton, NJ (US); Boris Mailhe, Plainsboro, NJ (US); Xiao Chen, Princeton, NJ (US); Mariappan S. Nadar, Plainsboro, NJ (US)

(21) Appl. No.: 16/688,170

(22) Filed: Nov. 19, 2019

(51) Int. Cl. G06T 11/00; G06N 3/08; G06N 3/04; G16H 30/40; G06T 7/00

(52) U.S. Cl. CPC G06T 11/008; G06N 3/088; G06N 3/04; G06T 2207/30004; G06T 7/0012; G06T 2207/10088; G06T 2207/20084; G16H 30/40

(57) ABSTRACT

For magnetic resonance imaging reconstruction, using a cost function independent of the ground truth and many samples of k-space measurements, machine learning is used to train a model with unsupervised learning. Due to use of the cost function with the many samples in training, ground truth is not needed. The training results in weights or values for learnable variables, which weights or values are fixed for later application. The machine-learned model is applied to k-space measurements from different patients to output magnetic resonance reconstructions for the different patients. The weights and/or values used are the same for different patients.

### FIELD

This disclosure relates to magnetic resonance (MR) imaging generally, and more specifically to MR reconstruction.

### BACKGROUND

MR imaging (MRI) is intrinsically slow, and numerous methods have been proposed to accelerate the MRI scan. Various types of MRI scans and corresponding reconstructions may be used. One acceleration method is the under-sampling reconstruction technique (i.e., MR compressed sensing), where fewer samples are acquired in the MRI data space (k-space), and prior knowledge is used to restore the images. An image regularizer is used in reconstruction to reduce aliasing artifacts. The MRI image reconstruction problem is often formulated as an optimization problem with constraints, and iterative algorithms, such as non-linear conjugate gradient (NLCG), fast iterated shrinkage/thresholding algorithm (FISTA), alternating direction method of multipliers (ADMM), Broyden-Fletcher-Goldfarb-Shanno (BFGS) quasi-Newton method, or the like, are used to solve the optimization problem.

Reconstruction is a processing intensive operation. Machine learning may be used to reduce processing for reconstruction for a given patient. Supervised deep learning (DL) approaches for MR reconstruction may be used but rely on datasets where ground truth (GT) is available. It is difficult (sometimes impossible) to acquire GT in the case of MR reconstruction because GT data relies on fully sampled data. Acquiring fully sampled MR data takes a long time and will then for most body regions be contaminated with motion artifacts.

### SUMMARY

By way of introduction, the preferred embodiments described below include methods, systems, instructions, and computer readable media for MRI reconstruction. Using a cost function independent of the GT and many samples of k-space measurements, machine learning is used to train a model with unsupervised learning. Due to use of the cost function with the many samples in training, GT is not needed. The training results in weights or values for learnable variables, which weights or values are fixed for later application. The machine-learned model is applied to k-space measurements from different patients to output MR reconstructions for the different patients. The weights and/or values used are the same for different patients.

In a first aspect, a method is provided for reconstruction of a magnetic resonance (MR) image in an MR system. The MR system scans a patient with an MR sequence. The scanning results in first k-space measurements. An image processor reconstructs the MR image from the first k-space measurements. The reconstruction is in response to input of the first k-space data to a deep machine-learned network. The deep machine-learned network has values for variables previously trained using unsupervised learning from multiple samples of second k-space measurements from patients, phantoms, and/or simulated MR, the previous training being from the samples without ground truths. The MR image is displayed.

In one embodiment, the scanning includes scanning with the MR sequence under sampling the patient. For example, MR-based compressive sensing is used.

A two-dimensional distribution of pixels representing an area of the patient are reconstructed. Alternatively or additionally, a three-dimensional distribution of voxels representing a volume of the patient are reconstructed. For voxels, volume or surface rendering from the voxels to a two-dimensional display is performed.

In one embodiment, the deep machine-learned network was previously trained with a GT independent cost function to avoid use of ground truth in machine training. For example, the cost function included a data fidelity term and a regularization term. The data fidelity term compares third k-space data transformed from object domain data output during machine learning to the second k-space data of the samples.

In other embodiments, reconstructing further includes inputting one or more MR system parameters with the first k-space measurements to the deep machine-learned network. The MR image is reconstructed from the first k-space measurements and the MR system parameters. Example MR system parameters include a coil sensitivity map and/or a bias field correction.

The scanning, reconstructing, and displaying may be repeated for a different patient. The reconstructing for the different patient applies the same values for variables of the deep machine-learned network.

In a second aspect, a method is provided for training a network for magnetic resonance (MR) reconstruction from signals collected by an MR scanner. A network is machine trained for the MR reconstruction with unsupervised deep machine learning. The machine training uses a plurality of samples of k-space data. The machine-learned network is stored as the network resulting from the machine training using the plurality of the samples, the machine-learned network having fixed weights determined based on the machine training.

In one embodiment, the machine training with the unsupervised deep machine learning includes machine training without ground truths for the samples. For example, unsupervised deep machine learning using training with a cost function, such as cost function having a regularization term and a data fidelity term. The data fidelity term is a difference of k-space information transformed from objects reconstructed from the samples and the k-space data of the samples. The regularization term is a variation in image domain data reconstructed from the k-space data of the samples.

In another embodiment, storing includes storing with the weights being trained weights of the network with the fixed weights comprises storing the network with the fixed weights being same weights having same values for application to k-space measurements from different patients.

In a third aspect, a system is provided for reconstruction in magnetic resonance (MR) imaging. An MR scanner is configured to scan a patient. The scan provides scan data in a scan domain. An image processor is configured to reconstruct a representation in an object domain from the scan data in the scan domain. The image processor is configured to reconstruct by application of the scan data to a machine-learned model. The machine-learned model has fixed weights from previous training. A display is configured to display an MR image from the reconstructed representation.

In one embodiment, the previous training of the machine-learned model was with unsupervised learning from a plurality of samples of k-space measurements and using a cost function without ground truths for the samples.

In another embodiment, the image processor is configured to reconstruct from the scan data and from values for one or more characteristics of the MR scanner in the scan of the patient.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

FIG. 1 is a block diagram of an embodiment of an MR system for medical imaging.

FIG. 2 is a flow chart diagram of one embodiment of a method for machine training for MR reconstruction.

FIG. 3 illustrates unsupervised training from multiple samples for MR reconstruction.

FIG. 4 is a flow chart diagram of one embodiment of a method for MR reconstruction using a machine-learned model previously trained in an unsupervised manner.

### DETAILED DESCRIPTION

Unsupervised learning of deep-learning networks is provided for MR reconstruction. Instead of relying on pairs of subsampled input k-space data and ground truth reconstructions from, for example, a full sampling k-space data, the goal is to train the network without ground truth data (i.e., train in an unsupervised way). Some approaches called “Deep Image Prior” (DIP) are unsupervised. In those non-MR approaches, the weights of the network are trained for each new image. As a result, these methods are very slow as opposed to the deep-learning approaches where the weights once learned are fixed for all the new tested images.

A cost function, such as a cost function used in DIP (e.g., data fidelity term plus a regularization term (e.g. 2D or 3D Total variation norm), is used as a loss function to machine train the MR reconstruction network. As opposed to DIP where the input of the network is a random vector and where the weights of the network are learned for each new image, the inputs for MR reconstruction are the k-space data (e.g., under sampled k-space data) with or without other additional data specific to the image being reconstructed (e.g. coil sensitivity maps, bias-field correction, etc.). As compared to the non-MR, non-MR reconstruction DIP methods, the weights of the network are fixed after training and during application, such as the same weights being used for different patients. As a consequence, the MR reconstruction is obtained via a simple path through the network so is fast.

Compared to classical deep-learning reconstruction using ground truth data, the machine learning for MR reconstruction is unsupervised and thus does not need labeled data (i.e. GT), which may be hard or impossible to obtain in some cases. Since a cost function, which does not require the GT, is used in training, largely available datasets may be used to train neural networks for reconstruction. The network is machine trained only once, and then all new MR measurements are processed by a simple path through the network. With DIP, training is required for each new set of input data.

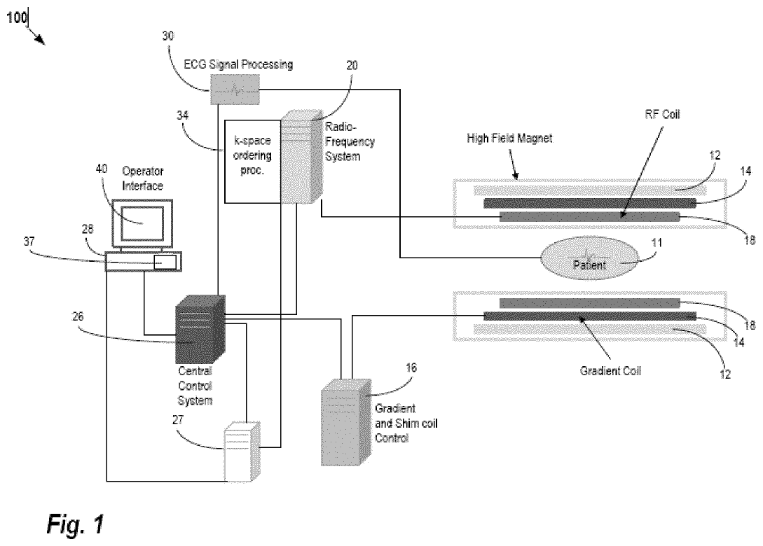

FIG. 1 shows one embodiment of a system for reconstruction in MRI.

The system uses a machine-learned model to reconstruct from k-space measurements for a patient to an object domain representation. Rather than data intensive optimization of fit between the representation and k-space measurements, the machine-learned model is used to output the representation in response to input of the k-space measurements. To machine train the model using more commonly available data, unsupervised learning is used. Many samples of k-space measurements from many scans are used to train with a (GT independent) cost function for adjusting the weights rather than comparison to an ideal reconstruction.

The system is implemented by an MR scanner or system, a computer based on data obtained by MR scanning, a server, or another processor. MR scanning system 100 is only exemplary, and a variety of MR scanning systems can be used to collect the MR data. In the embodiment of FIG. 1, the system is or includes the MR scanner or MR system 100. The MR scanner 100 is configured to scan a patient. The scan provides scan data in a scan domain. Frequency domain components representing MR scan data are acquired. The system 100 scans a patient to provide k-space measurements (measurements in the frequency domain), which may be stored in a k-space storage array. In the system 100, magnetic coils 12 create a static base magnetic field in the body of patient 11 to be positioned on a table and imaged. Within the magnet system are gradient coils 14 for producing position dependent magnetic field gradients superimposed on the static magnetic field. Gradient coils 14, in response to gradient signals supplied thereto by a gradient and shim coil control module 16, produce position dependent and shimmed magnetic field gradients in three orthogonal directions and generates magnetic field pulse sequences. The shimmed gradients compensate for inhomogeneity and variability in an MR imaging device magnetic field resulting from patient anatomical variation and other sources. The magnetic field gradients include a slice-selection gradient magnetic field, a phase-encoding gradient magnetic field, and a readout gradient magnetic field that are applied to patient 11.

RF (radio frequency) module 20 provides RF pulse signals to RF coil 18, which in response produces magnetic field pulses that rotate the spins of the protons in the imaged body of the patient 11 by ninety degrees, by one hundred and eighty degrees for so-called “spin echo” imaging, or by angles less than or equal to 90 degrees for so-called “gradient echo” imaging. Gradient and shim coil control module 16 in conjunction with RF module 20, as directed by central control unit 26, control slice-selection, phase-encoding, readout gradient magnetic fields, radio frequency transmission, and magnetic resonance signal detection, to acquire magnetic resonance signals representing planar slices of patient 11.

In response to applied RF pulse signals, the RF coil 18 receives MR signals, i.e., signals from the excited protons within the body as they return to an equilibrium position established by the static and gradient magnetic fields. The MR signals are detected and processed by a detector within RF module 20 and k-space component processor unit 34 to provide an MR dataset to an image data processor for processing into an image (i.e., for reconstruction in the object domain from the k-space data in the scan domain). In some embodiments, the image data processor is located in or is the central control unit 26. However, in other embodiments, such as the one depicted in FIG. 1, the image data processor is located in a separate unit 27. ECG synchronization signal generator 30 provides ECG signals used for pulse sequence and imaging synchronization. A two- or three-dimensional k-space storage array of individual data elements in k-space component processor unit 34 stores corresponding individual frequency components comprising an MR dataset. The k-space array of individual data elements has a designated center, and individual data elements individually have a radius to the designated center.

A magnetic field generator (comprising coils 12, 14 and 18) generates a magnetic field for use in acquiring multiple individual frequency components corresponding to individual data elements in the storage array. The individual frequency components are successively acquired using a Cartesian acquisition strategy as the multiple individual frequency components are sequentially acquired during acquisition of an MR dataset representing an MR image. A storage processor in the k-space component processor unit 34 stores individual frequency components acquired using the magnetic field in corresponding individual data elements in the array. The row and/or column of corresponding individual data elements alternately increases and decreases as multiple sequential individual frequency components are acquired. The magnetic field acquires individual frequency components in an order corresponding to a sequence of substantially adjacent individual data elements in the array, and magnetic field gradient change between successively acquired frequency components is substantially minimized. The central control processor 26 is programmed to sample the MR signals according to a predetermined sampling pattern. Any MR scan sequence may be used, such as for T1, T2, or other MR parameter. In one embodiment, compressive sensing scan sequence is used.

The central control unit 26 also uses information stored in an internal database to process the detected MR signals in a coordinated manner to generate high quality images of a selected slice(s) of the body (e.g., using the image data processor) and adjusts other parameters of system 100. The stored information comprises predetermined pulse sequence and magnetic field gradient and strength data as well as data indicating timing, orientation and spatial volume of gradient magnetic fields to be applied in imaging.

The central control unit 26 and/or processor 27 is an image process that reconstructs a representation of the patient from the k-space data. The image processor is a general processor, digital signal processor, three-dimensional data processor, graphics processing unit, application specific integrated circuit, field programmable gate array, artificial intelligence processor, digital circuit, analog circuit, combinations thereof, or other now known or later developed device for MR reconstruction. The image processor is a single device, a plurality of devices, or a network. For more than one device, parallel or sequential division of processing may be used. Different devices making up the image processor may perform different functions, such as reconstructing by one device and volume rendering by another device. In one embodiment, the image processor is a control processor or other processor of the MR scanner 100. Other image processors of the MR scanner 100 or external to the MR scanner 100 may be used. The image processor is configured by software, firmware, or hardware to reconstruct.

The image processor operates pursuant to stored instructions to perform various acts described herein. The image processor is configured by hardware, firmware, and/or software.

The image processor is configured to reconstruct a representation in an object domain. The object domain is an image space and corresponds to the spatial distribution of the patient. A planar area or volume representation is reconstructed. For example, pixels values representing tissue in an area or voxel values representing tissue distributed in a volume are generated.

The representation in the object domain is reconstructed from the scan data in the scan domain. The scan data is a set or frame of k-space data from a scan of the patient. The k-space measurements resulting from the scan sequence are transformed from the frequency domain to the spatial domain in reconstruction. In general, reconstruction is an iterative process, such as a minimization problem. This minimization can be expressed as:

$$x = \begin{matrix}arg min \\ x\end{matrix} {‖ Ax - y ‖}_{2}^{2} + λ {‖ Tx ‖}_{1} \qquad ( 1 )$$

where x is the target image to be reconstructed, and y is the raw k-space data. A is the MRI model to connect the image to MRI-space (k-space), which can involve a combination of an under-sampling matrix U, a Fourier transform F, and sensitivity maps S. T represents a sparsifying (shrinkage) transform. λ is a regularization parameter. The first term of the right side of equation 1 represents the image (2D or 3D spatial distribution or representation) fit to the acquired data, and the second term of the right side is a term added for denoising by reduction of aliasing artifacts due to under sampling. The 11 norm is used to enforce sparsity in the transform domain. ∥Ax−y∥₂² is the I2 norm of the variation of the under-sampled k-space data. Generally, the Pi norm is

$$\sqrt[p]{Σ {\vert x \vert}^{p}} .$$

In some embodiments, the operator T is a wavelet transform. In other embodiments, the operator T is a finite difference operator in the case of Total Variation regularization.

The image processor is configured to reconstruct by application of the scan data to a machine-learned model instead of performing the iterative reconstruction. Rather than performing the time and process intensive iterative optimization, the k-space measurements for a scan of a given patient are input to the machine-learned model. The machine-learned model outputs the reconstructed representation. The learned knowledge from machine learning is represented by values for learnable parameters or variables of the model. These values of the model are used to transform any k-space frame of data for a patient scan to a representation.

Machine learning is an offline training phase where the goal is to identify an optimal set of values of parameters of the model that can be applied to many different inputs (i.e., k-space measurements from patients). These machine-learned computed parameters can subsequently be used during clinical operation to rapidly reconstruct images. Once learned, the machine-learned model is used in an online processing phase in which new MR scan data for patients is input and the reconstructed representations for the patients are output based on the model values learned during the training phase.

During application to one or more different patients and corresponding different scan data, the same weights or values are used. The model and values for the learnable parameters are not changed from one patient to the next, at least over a given time (e.g., weeks, months, or years) or given number of uses (e.g., tens or hundreds). These fixed values and corresponding fixed model are applied sequentially and/or by different processors to scan data for different patients. Similarly, the same weights or values are used from beginning to end for reconstructing for a given patient rather than varying the weights or values as part of application. The model may be updated, such as retrained, or replaced but does not learn new values as part of application for a given patient.

The model has an architecture. This structure defines the learnable variables and the relationships between the variables. In one embodiment, a neural network is used, but other networks may be used. For example, a convolutional neural network (CNN) is used. Any number of layers and nodes within layers may be used. A DenseNet, U-Net, encoder-decoder, or another network may be used. Any know known or later developed neural network for reconstruction may be used.

Deep learning is used to train the model. The training learns both the features of the input data and the conversion of those features to the desired output (i.e., reconstructed representation) Backpropagation, RMSprop, ADAM, or another optimization is used. Since the training is unsupervised, the differences between the estimated reconstruction and the ground truth reconstruction are not minimized since ground truth is not provided. Instead, a GT independent cost function is used. The cost function measures some characteristic of the estimated reconstruction. The characteristic is one that likely distinguishes between good and bad reconstructions by examining the reconstruction rather than by comparison to a known good reconstruction. One or more terms may be used in the cost function, such as a cost function based on two or more characteristics of the estimated reconstruction.

The training uses multiple samples of input sets of k-space measurements. The scan data for these samples is generated by scanning a patient and/or phantom with different settings or sequences, scanning different patients and/or phantoms with the same or different settings or sequences, and/or simulating MR scanning with an MR scanner model. By using many samples, the model is trained to reconstruct given a range of possible inputs. The samples are used in deep learning to determine the values of the learnable variables (e.g., values for convolution kernels) that produce reconstructed representations with minimized cost function across the variance of the different samples.

The machine-learned model may have been trained to operate with other inputs in addition to the k-space measurements. For example, values for one or more characteristics of the MR scanner 100 as used in the scan of the patient may be input with the scan data. One or more coil sensitivity maps, one or more bias-field corrections, shimming, scan sequence used, and/or another characteristic of the MR scanner as used is input. The model is trained to reconstruct based on the input scan data and the characteristics of the system used to acquire the scan data.

Once trained, the machine-learned model reconstructs a spatial representation from input k-space measurements for a patient. The image processor may be configured to generate an MR image from the reconstructed representation. Where the representation is of an area, the values of the representation may be mapped to display values (e.g., scalar values to display color values) and/or formatted (e.g., interpolated to a display pixel grid). Alternatively, the output representation is of display values in the display format. Where the representation is of a volume, the image processor performs volume or surface rendering to render a two-dimensional image from the voxels of the volume. This two-dimensional image may be mapped and/or formatted for display as an MR image. Any MR image generation may be used so that the image represents the measured MR response from the patient.

Generated images of the reconstructed representation for a given patient are presented on display 40 of the operator interface. Computer 28 of the operator interface includes a graphical user interface (GUI) enabling user interaction with central control unit 26 and enables user modification of magnetic resonance imaging signals in substantially real time. Display processor 37 processes the magnetic resonance signals to provide image representative data for display on display 40, for example.

The display 40 is a CRT, LCD, plasma, projector, printer, or other display device. The display 40 is configured by loading an image to a display plane or buffer. The display 40 is configured to display the reconstructed MR image.

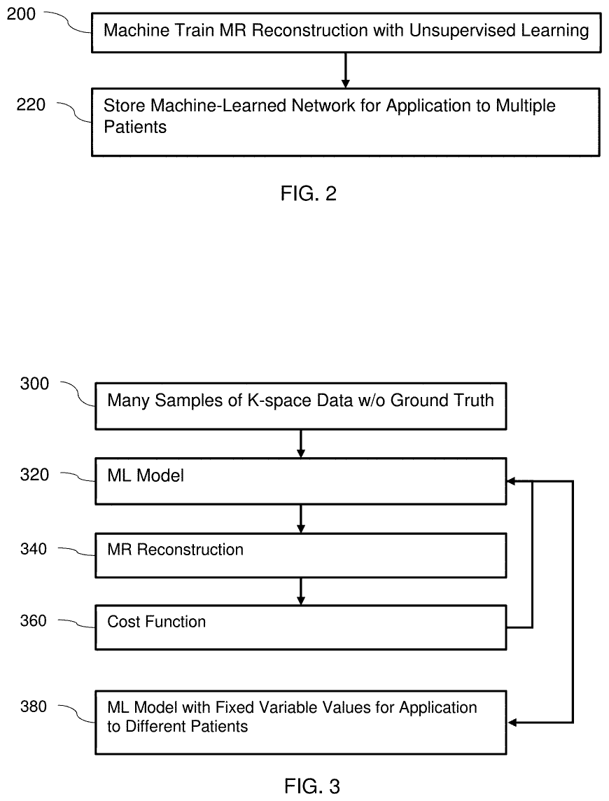

FIG. 2 is a flow chart diagram of one embodiment of a method for training a network for MR reconstruction, such as training to reconstruct from signals collected by an MR scanner. The method is to train using unsupervised machine learning where a GT independent cost function rather than ground truth is used with many input samples to learn to generate a reconstructed representation from input MR measurements. Once trained, the machine-learned model may be used with the same learned values to reconstruct representations of any number of patients from a respective number of sets of MR scan data for the patients.

The method is implemented by a computer, such as a personal computer, workstation, and/or server. Other computers may be configured to perform the acts of FIG. 2. The MR scanner 100 or central control unit 26 may implement the method. In one embodiment, the computer and a database are used to machine train and store the samples and trained model. The stored model is then distributed to one or more MR scanners 100 for application using the model as fixed (i.e., the learned values of the variables are not changed for reconstructions for a given patient and/or for different patients).

The method is performed in the order shown (i.e., top to bottom or numerical). Additional, different, or fewer acts may be provided. For example, instead of or in addition to storing in act 220, the machine-learned model is applied to previously unseen scan data for a patient to generate a reconstruction. As another example, acts for gathering and/or accessing training data are performed.

In act 200, a computer (e.g., image processor) machine trains a model for MR reconstruction. To machine train, training data is gathered or accessed. To machine learn, the training data includes many sets of MR scan data (e.g., k-space measurements). Tens, hundreds, or thousands of sample MR scans are acquired, such as from scans of patients, scans of phantoms, simulation of MR scanning, and/or by image processing to create further samples. Many examples that may result from different scan settings, patient anatomy, MR scanner characteristics, or other variance that results in different samples in MR scanning are used. In one embodiment, the samples are for MR compressed sensing, such as under sampled k-space data.

The training data may include other information for each sample. For example, the MR scanner characteristics are included. The coil sensitivity, bias-field correction, or other information for the MR scanner and corresponding scan for the patient may be included.

The training data does not include ground truth information. The desired representation or image resulting from a given sample is not provided. Instead, the machine learning uses minimization of a GT independent cost function (e.g., maximization of a reward function). Since known accurate ground truth reconstructions may be difficult to obtain, the cost function is used in training in an unsupervised manner.

Any GT independent cost function may be used. In one embodiment, the cost function is one used for the deep image prior approach for training as part of image generation for non-MR purposes. The cost function includes one or more terms. Each term reflects a different characteristic of the reconstructed representation.

In one embodiment, the cost function includes a regularization term and a data fidelity term. The data fidelity term measures a consistency with known information, such as the k-space data of the sample. An estimated representation (i.e., current model in training reconstructing from an input sample) is transformed back to the frequency or k-space domain. The resulting k-space measurements from the transform are compared to the k-space measurements of the input sample. The difference, such as an L₂ norm, is calculated. Other difference functions may be used. This distance or difference represents a cost. Greater difference provides greater cost as the reconstruction is less accurate. By finding the difference of k-space information transformed from objects reconstructed from the samples from the k-space data of the samples, the values of the learnable parameters may be altered to minimize this difference without needing ground truth difference.

The regularization term measures a characteristic of the reconstructed representation. For example, a good reconstruction in the image or object domain typically has less variation as compared to a poor reconstruction. The regularization may measure the amount of variation between pixels or voxels or in a given kernel. For example, a two or three-dimensional total variational norm is calculated as the regularization term. L₁ norm or other variation may be used. Other regularization, such as dynamic range, gradients, median, mean, or another statistic of the values of the representation, may be used.

Any architecture or layer structure for machine learning may be used. The architecture defines the structure, learnable parameters, and relationships between parameters. In one embodiment, a convolutional or another neural network is used. Deep machine training is performed. Any number of hidden layers may be provided between the input layer and output layer. The unsupervised training for MR reconstruction using input samples of MR scan data is independent of the network architecture, so may work with any reconstruction network.

For machine training, the model (e.g., network or architecture) is trained for MR reconstruction with unsupervised deep machine learning. An optimization, such as Adam, is performed using the various samples and cost functions. The values of the learnable parameters that minimize the cost function across the training samples are found using the optimization. The machine learns from the training data. The broad range of multiple examples of k-space measurements is used to learn.

FIG. 3 illustrates the training. The many samples 300 of k-space data without ground truth are used with the machine learning model 320. The machine learning model 320 is trained to generate MR reconstructions 340. The cost function 360 from the MR reconstruction 340 is used to determine a cost for each reconstruction from each sample 300 of the training data. The cost from the cost function is to be minimized, so one or more values of the learnable parameters are altered based on the cost in the feedback from the cost function 360 to the machine learning model 320. After many iterations, the cost is minimized. The resulting model 320 is a trained machine-learned model 380, which has values for learnable variables that are fixed for later application to different patients.

After training, the machine-learned model is represented as a matrix, filter kernels, and/or architecture with the learned values. The learned convolution kernels, weights, connections, and/or layers of the neural network or networks are provided.

In act 220 of FIG. 2, the computer or image processor stores the machine-learned neural network or other model resulting from the machine learning. The matrix or other parameterization of the machine-learned model are saved in memory. The machine-learned neural network may be stored locally or transferred over a network or by moving the memory to other computers, workstations, and/or MR scanners.

The network or other model resulting from the machine training using the plurality of the samples is stored. This stored model has fixed weights or values of learnable parameters determined based on the machine training. These weights or values are not altered by patient-to-patient or over multiple uses for different MR scans. The weights or values are fixed, at least over a number of uses and/or patients. The same weights or values are used for different sets of MR scan data corresponding to different patients. The same values or weights may be used by different MR scanners. The fixed machine-learned model is to be applied without needing to train as part of the application. Random initialization as part of reconstruction for a given patient is not needed as the fixed values or weights are instead used.

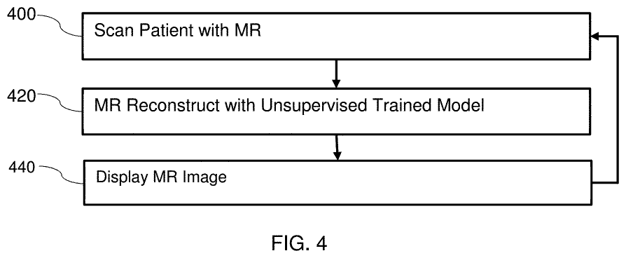

FIG. 4 is a flow chart diagram of one embodiment of a method for reconstruction of a MR image in an MR system. A machine-learned model as trained is applied. The machine-learned model, having been trained in an unsupervised manner, generates a MR image as a reconstruction or representation from k-space data measured for a patient. Having been trained in an unsupervised manner, the machine-learned model generates the MR image in a learned manner different than if trained with ground truth. The values of the model used in application are different due to unsupervised training than where supervised training had been used.

The method is performed by the system of FIG. 1 or another system. The MR scanner scans the patient. An image processor reconstructs the MR image using the machine-trained network, and a display displays the MR image. Other components may be used, such as a remote server or a workstation performing the reconstruction and/or display.

The method is performed in the order shown or other orders. Additional, different, or fewer acts may be provided. For example, a preset, default, or user input settings are used to configure the scanning prior art act 400. As another example, the MR image is stored in a memory (e.g., computerized patient medical record) or transmitted over a computer network instead of or in addition to the display of act 440.

In act 400, the MR system scans a patient with an MR sequence. For example, the MR scanner or other MR system scans the patient with an MR compressed (e.g., under sampling) or another MR sequence. The amount of under sampling is based on the settings, such as the acceleration. Based on the configuration of the MR scanner, a pulse sequence is created. The pulse sequence is transmitted from coils into the patient. The resulting responses are measured by receiving radio frequency signals at the same or different coils. The scanning results in k-space measurements as the scan data. These k-space measurements for a given patient are new and/or not included in the samples for training.

In act 420, an image processor reconstructs the MR image from the k-space measurements of the patient. For reconstruction, the k-space data is Fourier transformed into scalar values representing different spatial locations, such as spatial locations representing a plane through or volume in the patient. Scalar pixel or voxel values are reconstructed as the MR image. The spatial distribution of MR measurements in object or image space is formed. This spatial distribution represents the patient.

The reconstruction is performed, at least in part, using a deep machine-learned network, such as a neural network trained with deep machine learning. The machine-learned network is previously trained, and then performs reconstruction as trained. Fixed values of learned parameters are used for application to the patient rather than inputting random noise and training as part of application for a given patient. The machine-learned network is previously trained, using many samples of input k-space measurements, to reconstruct.

In application, the values for the variables previously trained using unsupervised learning are applied in the deep machine-learned network. Multiple input samples of k-space measurements from patients, phantoms, and/or simulated MR are used to train. Rather than comparison with ground truth, a GT independent cost function was used in the training. A calculatable characteristic or characteristics of the estimated reconstructions from the sample inputs is used in training. For example, the cost function includes a data fidelity term and a regularization term. In one embodiment, the cost function (e.g., data fidelity term plus a regularization term (e.g. 2D or 3D Total variation norm)) used in Deep Image Prior is used to train the reconstruction network. The data fidelity term compares k-space measurements, one from the input sample and the other transformed from the network estimated reconstruction (e.g., transform from object domain output during training back to the Fourier or k-space domain). The difference is to be minimized. The training occurred without ground truths, avoiding the difficulty in finding sufficient samples with ground truth.

Once trained, the weights or values of the learnable parameters of the machine-learned network are fixed or held constant over one or multiple ruses. As opposed to generating an image using randomized initialization and/or training as part of application for a given image, the previously learned values or weights are used without change. For all new tests or applications, the same values or weights are used. As a consequence, the reconstruction occurs quickly, such as within seconds, of input of the k-space measurements.

In application of the already trained network, the k-space data for the patient is input to the machine-learned network. The measurements from a complete MR scan (e.g., from a compressed sensing scan) of a given patient are input. As opposed to input of a random vector and learning weights of the network for each new image or patient in application, the inputs are the under sampled or other k-space measurements.

Other data may be input, such as MR scanner settings or characteristics. For example, one or more MR system parameters as set, calibrated, configured, or used are input with the k-space measurements to the deep machine-learned network. Any setting or characteristic of the MR system may be input, such as the coil sensitivity map(s), bias field correction(s), shimming setting(s), scan sequence identity, motion information, or other information. Additional data specific to the image being reconstructed is input with the k-space measurements. The MR image is reconstructed from the k-space measurements and the MR system parameters.

In response to the input for a given patient, a patient specific MR image is reconstructed. The machine-learned network outputs the MR image as pixels, voxels, and/or a display formatted image in response to the input. The learned values and network architecture determine the output from the input. The output of the machine-learned network is a two-dimensional distribution of pixels representing an area of the patient and/or a three-dimensional distribution of voxels representing a volume of the patient.

Other processing may be performed on the input k-space measurements before input. Other processing may be performed on the output representation or reconstruction, such as spatial filtering, color mapping, and/or display formatting. In one embodiment, the machine-learned network outputs voxels or scalar values for a volume spatial distribution as the MR image. Volume rendering is performed to generate a display image as a further MR image. In alternative embodiments, the machine-learned network outputs the display image directly in response to the input.

In act 440, a display (e.g., display screen) displays the MR image. The MR image is formatted for display on the display. The display presents the MR image for viewing by the user, radiologist, physician, clinician, and/or patient. The image is rapidly generated from the k-space measurements and assists in diagnosis.

The displayed image may represent a planar region or area in the patient. Alternatively or additionally, the displayed image is a volume or surface rendering from voxels (three-dimensional distribution) to the two-dimensional display.

The feedback from act 440 to act 400 represents repetition of application using the same machine-learned network. The same deep machine-learned network may be used for different patients. The different k-space measurements from the different patients are used with the same machine-learned network. The same or different copies of the same machine-learned network are applied for different patients, resulting in reconstruction of patient-specific representations or reconstructions using the same values or weights of the learned parameters of the network. Different patients and/or the same patient at a different time may be scanned while the same or fixed trained reconstruction network reconstructs the MR image. Other copies of the same deep machine-learned neural network may be used for other patients with the same or different scan settings and corresponding sampling or under sampling in k-space. The scanning of act 400, the reconstructing of act 420, and displaying of act 440 are repeated for a different patient and/or for a different scan. The reconstructing of act 420 for the different patient or scan applies the same values for variables of the deep machine-learned network.

Although the subject matter has been described in terms of exemplary embodiments, it is not limited thereto. Rather, the appended claims should be construed broadly, to include other variants and embodiments, which can be made by those skilled in the art.

What is claimed is:

1. A method for reconstruction of a magnetic resonance (MR) image in an MR system, the method comprising:
scanning, by the MR system, a patient with an MR sequence, the scanning resulting in first k-space measurements;
reconstructing, by an image processor, the MR image from the first k-space measurements, the reconstructing inputting the first k-space data to a deep machine-learned network, the deep machine-learned network applying values for variables previously trained using unsupervised learning from multiple samples of second k-space measurements from patients, phantoms, and/or simulated MR, the previous training being from the samples without ground truths; and
displaying the MR image.

2. The method of claim 1 wherein scanning comprises scanning with the MR sequence under sampling the patient.

3. The method of claim 1 wherein reconstructing comprises reconstructing a two-dimensional distribution of pixels representing an area of the patient.

4. The method of claim 1 wherein reconstructing comprises reconstructing a three-dimensional distribution of voxels representing a volume of the patient, and wherein displaying comprises volume or surface rendering from the voxels to a two-dimensional display.

5. The method of claim 1 wherein reconstructing comprises reconstructing with the deep machine-learned network having been previously trained with a cost function that did not depend on the ground truth.

6. The method of claim 5 wherein reconstructing comprises reconstructing with the deep machine-learned network having been previously trained with the cost function, the cost function including a data fidelity term and a regularization term.

7. The method of claim 5 wherein reconstructing comprises reconstructing with the deep machine-learned network having been previously trained with the cost function, the data fidelity term comparing third k-space data transformed from object domain data output during machine learning to the second k-space data of the samples.

8. The method of claim 1 wherein reconstructing further comprises inputting one or more MR system parameters with the first k-space measurements to the deep machine-learned network, the MR image reconstructed from the first k-space measurements and the MR system parameters.

9. The method of claim 8 wherein inputting comprises inputting a coil sensitivity map and/or a bias field correction as the MR system parameters.

10. The method of claim 1 further comprising repeating the scanning, reconstructing, and displaying for a different patient, wherein the reconstructing for the different patient applies the same values for variables of the deep machine-learned network.

11. A method for training a network for magnetic resonance (MR) reconstruction from signals collected by an MR scanner, the method comprising:
machine training a network for the MR reconstruction with unsupervised deep machine learning, the machine training using a plurality of samples of k-space data; and
storing a machine-learned network as the network resulting from the machine training using the plurality of the samples, the machine-learned network having fixed weights determined based on the machine training.

12. The method of claim 11 wherein machine training with the unsupervised deep machine learning comprises machine training without ground truths for the samples.

13. The method of claim 11 wherein machine training with the unsupervised deep machine learning comprises machine training with a cost function that does not depend on the ground truth.

14. The method of claim 13 wherein machine training with the cost function comprises training with the cost function comprising a regularization term and a data fidelity term.

15. The method of claim 14 wherein machine training comprises machine training with the data fidelity term being a difference of k-space information transformed from objects reconstructed from the samples and the k-space data of the samples.

16. The method of claim 14 wherein machine training comprises machine training with the regularization term being a variation in image domain data reconstructed from the k-space data of the samples.

17. The method of claim 11 wherein storing comprises storing with the weights comprises trained weights of the network with the fixed weights comprises storing the network with the fixed weights being same weights having same values for application to k-space measurements from different patients.

18. A system for reconstruction in magnetic resonance (MR) imaging, the system comprising:
an MR scanner configured to scan a patient, the scan providing scan data in a scan domain;
an image processor configured to reconstruct a representation in an object domain from the scan data in the scan domain, the image processor configured to reconstruct by application of the scan data to a machine-learned model, the machine-learned model having fixed weights from previous training; and
a display configured to display an MR image from the reconstructed representation.

19. The system of claim 18 wherein the previous training of the machine-learned model was with unsupervised learning from a plurality of samples of k-space measurements and using a cost function without ground truths for the samples.

20. The system of claim 18 wherein the image processor is configured to reconstruct from the scan data and from values for one or more characteristics of the MR scanner in the scan of the patient.
